(19) United States
(12) Patent Application Publication
(10) Pub. No.: US 2009/0182058 A1
(43) Pub. Date: Jul. 16, 2009

(54) Novel Chalcone Derivatives With Antimitotic Activity

(76) Inventors: Ahcene Boumendjel, Meylan (FR); Charles Dumontet, Venissieux (FR); Madeleine Blanc, Saint-Jean De Moirans (FR); Anne-Marie Mariotte, Saint Simeon De Bressieux (FR)

Correspondence Address: CLARK & BRODY, 1090 VERMONT AVENUE, NW, SUITE 250, WASHINGTON, DC, 20005, US

(21) Appl. No.: 12/087,790

(22) PCT Filed: Jan. 15, 2007
(86) PCT No.: PCT/FR2007/050654

§ 371 Date: Nov. 17, 2008

(30) Foreign Application Priority Data
Jan. 17, 2006 (FR) 0600389

(51) Int. Cl. A61K 31/12; C07C 49/255; A61P 35/00; A61P 37/00

(52) U.S. Cl. 514/685; 568/325

(57) ABSTRACT

The present invention relates to novel chalcone derivatives of formula (I):

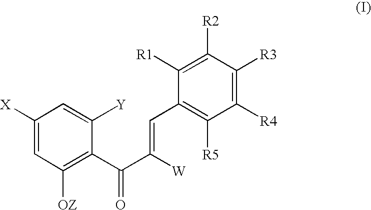

wherein X, Y, Z, W, RI, R2, R3, R4 and R5 are as defined, said derivatives having antimitotic activity, as well as to pharmaceutical compositions containing such compounds and to their use for making drugs.

The present invention relates to novel chalcone derivatives having antimitotic activity, as well as to pharmaceutical compositions containing such compounds and to their use for making drugs.

Many studies have been conducted on chalcone derivatives. As an illustration, mention may be made of the application U.S. Pat. No. 6,462,075 which describes chalcone derivatives having angiogenesis inhibitor activity. These compounds are shown as being able to be used as anti-tumoral, anti-cancer agents, for treating angiogenic diseases of the skin and chronic inflammatory diseases.

According to the present invention, the inventors have developed a novel series of chalcones having dose-response properties in flow cytometry on several tumoral lines and in cytotoxicity, improved relatively to the compounds of the prior art with the closest structures.

The object of the present invention is in particular novel chalcone derivatives of formula (I):

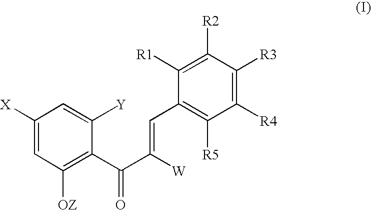

X represents a hydrogen atom or an —OH, —O-methyl, —O-ethyl, —O-propyl, —O-isopropyl, —O-benzyl, —NH₂, —NHCORₐ group

with Rₐ which is a linear or branched alkyl group with 1 to 5 carbon atoms, an aryl group which may be mono- or poly-substituted with a substituent selected from halogen atoms, OH, OMe and —NR_{b}R_{c} groups with R_{b} and R_{c} representing independently of each other, a linear or branched alkyl chain with 1 to 6 carbon atoms,

Y represents a hydrogen atom or an —OH, —O-methyl, —O-ethyl, —O-propyl, —O-isopropyl, —O-benzyl group,

it being understood that at least one of the X and Y groups is different from hydrogen,

Z represents a hydrogen atom or a methyl, ethyl, propyl, isopropyl, benzyl group,

W represents a hydrogen atom, or an —OH, —O-methyl, —O-ethyl, or —O-benzyl group,

R1, R2, R3, R3, R4 and R5 represent independently of each other a hydrogen or halogen atom, or an —OH, methyl, —O-methyl, ethyl, —O-ethyl, propyl, —O-propyl, benzyl, —O-benzyl, —NH₂, —NHCH₃, —N(CH₃)₂, it being understood that at least two substituants R1, R2, R3, R4 and R5 are different from H, and that when X or Y represents a hydrogen atom, at least three substituants R1, R2, R3, R4 and R5 are different from H,

as well as their pharmaceutically acceptable, hydrates, solvates or salts, except for 2′-hydroxy-4′,6′,2,4,5-pentamethoxychalcone, and 2′,4′,6′,2,4,5,6-pentamethoxy-chalcone.

In all the compounds of formula (I), the configuration around the double bond α,β is trans as indicated on the structural formula (I).

In the above definition, by halogen is meant a chlorine, bromine, iodine or fluorine atom.

By alkyl is meant a saturated hydrocarbon chain.

By aryl is meant a phenyl, naphthyl, or cinnamyl group.

According to preferred aspects of the invention, the compounds of formula (I) have any of the features hereafter or a combination of several of these features, when they are not mutually exclusive:

at least three of the substituants R1, R2, R3, R4 and R5 are different from hydrogen,

X represents a hydrogen atom or an —OMe, —OEt, —NH₂ group, the OMe group being preferred, it being understood that when X represents a hydrogen atom, Y represents an —OH, —O-methyl, —O-ethyl, —O-propyl, —O-isopropyl, —O-benzyl group,

Y represents a hydrogen atom or an —OMe, —OEt group, the OMe group being preferred, it being understood that, when Y represents a hydrogen atom, X represents an —OH, —O-methyl, —O-ethyl, —O-propyl, —O-isopropyl, —O-benzyl group,

Z represents a hydrogen atom or a methyl or ethyl group,

W represents an -O-methyl group or preferably a hydrogen atom,

R1, R2, R3, R4 and R5 represent independently of each other, a hydrogen or fluorine atom, a methyl, —O-methyl, —O-ethyl group, it being understood that at least two substituants RI1, R2, R3, R4 and R5 are different from H, and that when X or Y represents a hydrogen atom, at least three substituants R1, R2, R3, R4 and R5 are different from H,

they fit formula (Ia):

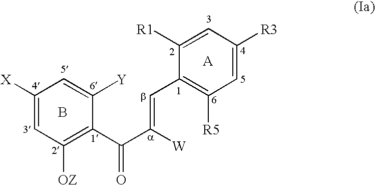

with X, Y, R1, R3 and R5 as defined earlier for (I).

The compounds of formula (I) hereafter as well as their pharmaceutically acceptable salts, hydrates or solvates, are more preferred:

2,2′,4,6,6′-pentamethoxychalcone:

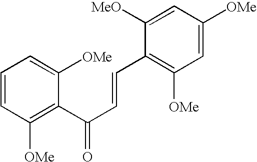

2,2′,4,4′,6,6′-hexamethoxychalcone:

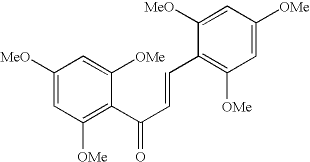

2,2′,4,4′,6-pentamethoxychalcone:

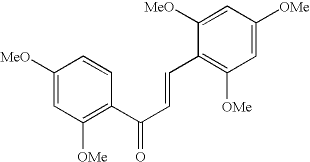

2′-hydroxy-2,4,4′,5-tetramethoxychalcone:

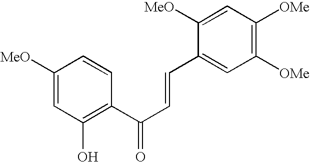

2′-hydroxy-2,4,4′,6-tetramethoxychalcone:

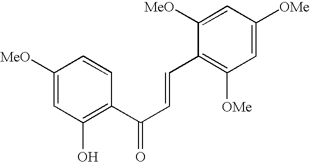

2′-hydroxy-3,4,4′,5-tetramethoxychalcone:

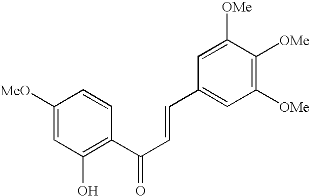

2′-hydroxy-2,3,4,4′-tetramethoxychalcone:

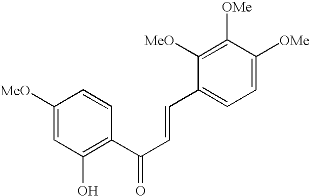

2′,6′-diethoxy-2,4,6-trimethoxychalcone:

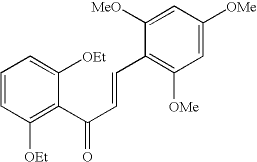

2′,6′-dimethoxy-2,4,6-triethoxychalcone:

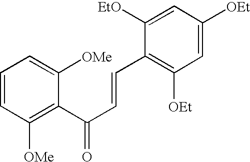

4′-amino-2,2′,4,6,6′-pentamethoxychalcone:

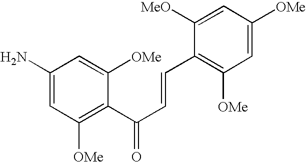

2,2′,4,4′,6,6′-hexaethoxychalcone:

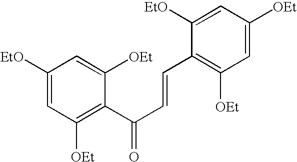

2,2′,3,4,6′-pentamethoxychalcone:

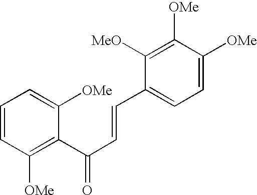

2,2′,4,5,6′-pentamethoxychalcone:

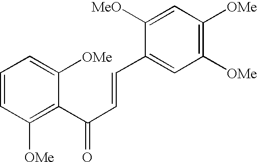

2′,3,4,5,6′-pentamethoxychalcone:

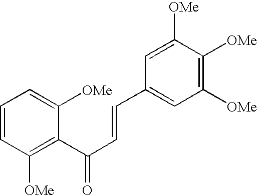

3,5-difluoro-2,2′,4,6,6′-pentamethoxychalcone:

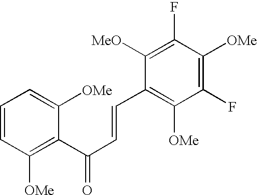

2′,6′-dimethoxy-2,4,6-trimethylchalcone:

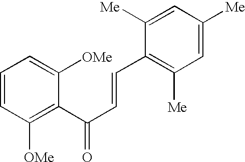

2,2′,4,4′,6′-pentamethoxychalcone:

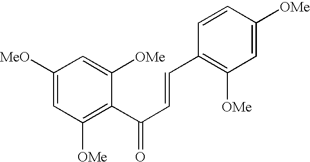

2,2′,4′,6,6′-pentamethoxychalcone:

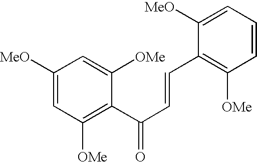

α,2,2′,4,4′,6,6′-heptamethoxychalcone:

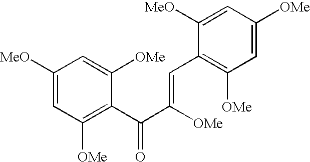

The study of the structure-activity relationship indicates that the nature of the substituants of the phenyl groups and their position are decisive for antimitotic activity.

On the ring A of the chalcone, a methoxy group seems to be the most advantageous among all the investigated substituants and the positions 2, 4 and 6 are the most important for the substitution.

In conclusion, di- or tri-methoxylation on the 2,4,6 carbon atoms of the ring A and on the 2′,4′,6′ carbon atoms of the ring B are particularly advantageous for antimitotic activity of the chalcones described in this invention.

The salts of the compounds according to the invention are prepared according to techniques well known to one skilled in the art. The salts of the compounds of formula (I) according to the present invention comprise those with mineral or organic acids which allow appropriate separation or crystallization of the compounds of formula (I), as well as of pharmaceutically acceptable salts. As an appropriate acid, mention may be made of: picric acid, oxalic acid, or an optically active acid, for example a tartaric acid, a dibenzoyltartaric acid, a mandelic acid or a camphosulfonic acid, and those which form physiologically acceptable salts, such as the hydrochloride, hydrobromide, sulphate, hydrogensulfate, dihydrogenphosphate, maleate, fumarate, 2-naphthalenesulfonate, paratoluenesulfonate.

When a compound according to the invention has one or more asymmetric carbon atoms, the optical isomers of this compound are an integral part of the invention. The present invention comprises the compounds of formula (I) as pure isomers but also as a mixture of isomers in any proportion. The compounds (I) are isolated as pure isomers by standard separation techniques; for example fractionated recrystallizations of a salt of the racemic with an optically active acid or base may be used, the principle of which is well known or the standard techniques of chromatographies on a chiral or non-chiral phase.

The functional groups possibly present in the molecule of the compounds of formula (I) and in the reaction intermediates may be protected, either in a permanent form or in a temporary form, by protective groups which provide one-to-one synthesis of the expected compounds. The protection and deprotection reactions are carried out according to techniques well known to one skilled in the art. By temporary protective group of amines, alcohols or carboxylic acids, are meant protective groups such as those described in “Protective Groups in Organic Synthesis”, Greene T. W. and Wuts P. G. M., ed. John Wiley and Sons, 1991 and in “Protecting Groups” Kocienski P. J., 1994, Georg Thieme Verlag.

The compounds of formula (I) according to the invention wherein W═H are obtained by aldolic condensation of an acetophenone of formula (II) and of a benzaldehyde of formula (III) as illustrated in Scheme 1 hereafter wherein X, Y, Z, R1, R2, R3, R4 and R5 are as defined for (I):

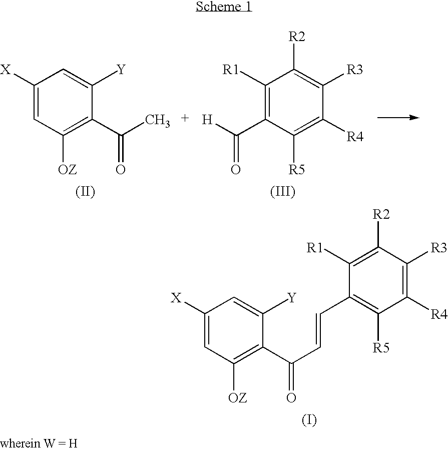

Such an aldolic condensation is conducted in a basic medium, for example in the presence of potash, preferably in a polar solvent such as methanol.

The compounds of formula (II) are commercial compounds or prepared according to techniques well known to one skilled in the art. For example, in the case when OZ, X, Y═OMe, the acetophenone (II) is prepared by methylation of hydroxylated acetophenone by using methyl sulphate or methyl iodide as methylation agents according to techniques well known to one skilled in the art.

In the case when X═NH_{2,} the acetophenone (II) may be prepared according to the method described by N. Deka; M. Hadjeri; M. Lawson; C. Beney; A-M. Mariotte and A. Boumendjel in “Acetylated dimethoxyaniline as a key intermediate for the synthesis of aminoflavones and quinolones”, Heterocycles 2002, 57, 123-128.

For compounds wherein W is a methoxy, acetophenone is obtained from phloroglucinol and from methoxyacetonitrile according to the method described by K. Wähälä and T. A. Hase in “Expedient synthesis of polyhydroxyisoflavones”, J. Chem. Soc., Perkin Trans. 1. 1991, 3005-3008. The compounds wherein W is a hydroxy, O-ethyl, or O-benzyl, acetophenone is obtained from phloroglucinol on the one hand and from hydroxyacetic acid, 2-ethyloxyacetic acid or 2-benzyloxyacetic acid respectively, according to the method described by Wähälä et al.

The compounds of formula (III) are commercial compounds.

No sign of toxicity is observed with these compounds at pharmacologically active doses and their toxicity is therefore compatible with their use as drugs.

In particular, the compounds of general formula (I) as defined earlier, or their pharmaceutically acceptable salts, may be used for preparing a drug intended for treating the following diseases/disorders/natural phenomena: tumoral proliferation and dissemination, excessive proliferation of normal cells, pathological angiogenesis, excessive activity of the immune system. The compounds according to the invention are particularly of interest for their antimitotic activity and may notably be used as antitumoral or anticancer agents. Generally, the compounds according to the invention may be used for preparing a drug intended for the treatment, as a curative or preventive treatment, of any type of cancer.

In particular, the compounds of formula (I), as well as their pharmaceutically acceptable salts, may be used for making drugs intended for treating or preventing benign tumoral lesions, immunity disorders characterized by an excessive activity of the immune system, pathologies characterized by excessive angiogenic activity, cancers, including malignant hemopathies and solid tumors, including tumors of glandular, mesenchymatous, genital, cutaneous and neurological origin.

As examples of such pathologies, mention may be made of benign tumoral lesions such as skin naevi, aerodigestive and mucosal polyps, adenomas; immunity disorders characterized by excessive activity of the immune system such as auto-immune diseases with auto-antibodies, non-exhaustively represented by rheumatoid arthritis, lupus, scleroderma, auto-immune thyroiditises, vitiligo, auto-immune cirrhoses, auto-immune pneumonias; secondary signs to hypersensitivity, for example rhinitis, allergic conjunctivitis and asthma, eczema, medication allergies; pathologies characterized by increased angiogenic activity non-exhaustively represented by diabetic retinopathy, angiomas.

In particular, mention may be made of cancers which may be treated with the compounds of the present invention, carcinomas, malignant hemopathies of myeloid and lymphoid lines, tumors of mesenchymatous origin, sarcomas, tumors of the central and peripheral nervous system, melanomas, seminomas, teratocarcinomas, osteosarcomas, xeroderma pigmentosum, kerato acanthoma, endocrine neoplasias, and Kaposi's sarcoma. Also, the object of the present invention is therefore the compounds of formula (I), as well as their pharmaceutically acceptable salts, or possibly solvates or hydrates, as drugs, pharmaceutical compositions containing an effective dose of a compound according to the invention or a pharmaceutically acceptable salt, solvate or hydrate of the latter, and appropriate excipients.

Said excipients are selected according to the pharmaceutical form and the desired administration method.

In the pharmaceutical compositions of the present invention for oral, sublingual, subcutaneous, intramuscular, intravenous, topical, intratracheal, intranasal, transdermal, rectal or intraocular administration, the active ingredients of formula (I) above or their possible salts, solvates and hydrates, may be administered as unit dosage forms, mixed with conventional pharmaceutical carriers, to animals and to humans for prophylaxy or treatment of the above disorders or diseases. The suitable unit administration forms comprise oral-route forms such as tablets, gelatine capsules, powders, granules and oral solutions or suspensions, sublingual, buccal, intratracheal, intranasal administration forms, subcutaneous, intramuscular or intravenous administration forms and rectal administration forms. For topical application, the compounds according to the invention may be used in creams, ointments, lotions or eye drops.

In order to obtain the desired prophylactic or therapeutic effect, each unit dose may contain from 0.1 to 10,000 mg of compound according to the invention in combination with a pharmaceutical carrier. This unit dose may be administered 1 to 5 times a day so as to administrate a daily dosage with which the desired effect may be obtained.

When a solid composition is prepared as tablets, the main active ingredient is mixed with a pharmaceutical carrier, such as gelatine, starch, lactose, magnesium stearate, talc, gum arabic or the like. The tablets may be coated with saccharose, with a cellulose derivative, or other suitable materials or further they may be treated so as to have prolonged or delayed activity, and continuously release a predetermined amount of active ingredient.

A gelatine capsule preparation is obtained by mixing the active ingredient with a diluent and by pouring the obtained mixture in soft or hard gelatine capsules.

The pharmaceutical compositions containing a compound of the invention may also appear in liquid form, for example as solutions, emulsions, suspensions or syrups. Suitable liquid carriers may for example be water, organic solvents such as glycerol or glycols, as well as their mixtures in various proportions with water.

A preparation as a syrup or an elixir or for administration as drops may contain the active ingredient together with a sweetener, preferably an acaloric sweetener, methylparaben and propylparaben as an antiseptic, as well as an agent providing taste and a suitable coloring agent. The water-dispersible powders or granules may contain the active ingredient mixed with dispersion agents or wetting agents or suspension agents, such as polyvinylpyrrolidone, just as with sweeteners or taste-correcting agents.

For rectal administration, one resorts to suppositories which are prepared with binders which melt at the rectal temperature, for example cocoa butter or polyethylene glycols. For parenteral administrations, aqueous suspensions, isotonic saline solutions or sterile and injectable solutions are used, which contain dispersion agents and/or pharmacologically compatible wetting agents, for example propylene glycol, or butylene glycol. The active ingredient may also be formulated as microcapsules, possibly with one or more carriers or additives, or else with matrices such as a polymer or a cyclodextrin (patch, prolonged release forms).

The compositions of the present invention may contain, in addition to the products of formula (I) above or their pharmaceutically acceptable salts, solvates and hydrates, for example active ingredients which may be useful in the treatment of the disorders or diseases indicated above.

Thus, the object of the present invention is also pharmaceutical compositions containing several active ingredients in association, one of which is a compound according to the invention.

The object of the invention is therefore their use as drugs which may be used alone or in combination with treatments such as chemotherapy, radiotherapy or anti-angiogenic treatments possibly applying other active substances.

Moreover, in a general way, the same preferences as those indicated earlier for the compounds of general formula (I) are applicable mutatis mutandis to drugs, pharmaceutical compositions and to a use applying the compounds according to the invention.

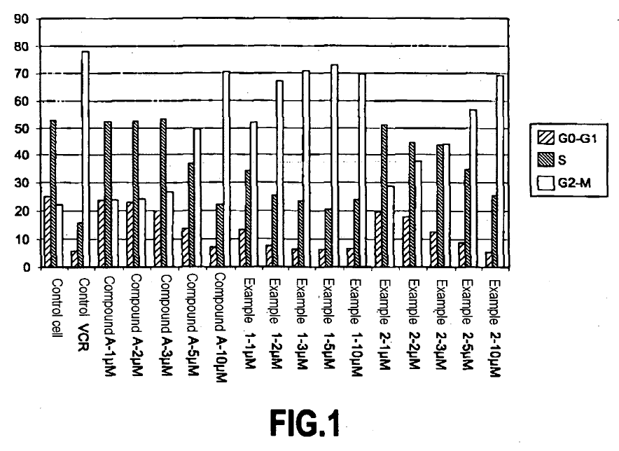

The examples hereafter, with reference to FIG. 1, are provided in order to illustrate the invention, but do not have any limiting character.

FIG. 1 shows the dose-response effect of the compounds of examples 1 and 2 and of the compound A on K562 cell cycle blocking.

Preparation of the compounds of formula (I):

### EXAMPLE 1

2,2′,4,6,6′-pentamethoxychalcone of formula:

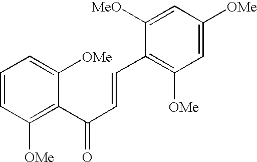

1 mM of 2,6-methoxyacetophenone and 1 mM of 2,4,6-trimethoxybenzaldehyde in 10 ml of methanol, in the presence of 1 ml of 25% KOH, were heated to 70° C. for 3 hours. The solvent is then evaporated under reduced pressure, water is added and the mixture is extracted with CH₂Cl₂. The organic phase is dried, evaporated. The obtained products are solids. Purification is carried out with chromatography by eluting with a (1:5) mixture of ethyl acetate and cyclohexane The chalcone is obtained with a yield of 24%. m.p.=170° C.

The Examples 2 to 22 provided in TABLE 1 hereafter were prepared likewise, in the presence of 1 ml of 50% KOH. Purification is carried out either by washing with ether (a), or by chromatography (b).

TABLE 1
Compounds of formula (I) wherein W = H

| Ex. | OZ | X | Y | R1 | R2 | R3 | R4 | R5 | purif. | m.p. ° C. | dt |
|---|---|---|---|---|---|---|---|---|---|---|---|
| 2 | OMe | OMe | OMe | OMe | H | OMe | H | OMe | a | 150 | 62 |
| 3 | OMe | OMe | H | OMe | H | OMe | H | OMe | b | 162 | 11 |
| 4 | OMe | OMe | OH | OMe | H | H | H | OMe | a | 152 | 58 |
| 5 | OMe | OMe | OH | H | OMe | H | OMe | H | a | 163 | 73 |
| 6 | OMe | OMe | OH | CI | H | H | H | 01 | b | 114 | 6 |
| 7 | OEt | H | OEt | OMe | H | OMe | H | OMe | b | 102 | 31 |
| 8 | OMe | H | OMe | OEt | H | OEt | H | OEt | b | 154 | 10 |
| 9 | OMe | NH2 | OMe | OMe | H | OMe | H | OMe | a | 188 | 72 |
| 10 | OEt | OEt | OEt | OEt | H | OEt | H | OEt | a | 160 | 21 |
| 11 | OMe | H | OMe | OMe | OMe | OMe | H | H | a | 129 | 75 |
| 12 | OMe | H | OMe | OMe | H | OMe | OMe | H | a | 143 | 75 |
| 13 | OMe | H | OMe | H | OMe | OMe | OMe | H | a | 136 | 84 |
| 14 | OMe | H | OMe | OMe | F | OMe | F | OMe | a | 108 | 40 |
| 15 | OMe | H | OMe | CH₃ | H | CH₃ | H | CH₃ | a | 126 | 74 |
| 16 | OMe | OMe | OMe | H | H | OMe | H | OMe | a | 105 | 67 |
| 18 | OMe | OMe | OMe | OMe | H | H | H | OMe | a | 164 | 81 |
| 19 | OH | OMe | H | OMe | H | OMe | OMe | H | a | 170 | 15 |
| 20 | OH | OMe | H | OMe | H | OMe | H | OMe | b | 167 | 26 |
| 21 | OH | OMe | H | H | OMe | OMe | OMe | H | a | 110 | 29 |
| 22 | OH | OMe | H | OMe | OMe | OMe | H | H | a | 152 | 76 |

For Example 14, 1 ml of potash (KOH) diluted to 25% is used as in Example 1.

In the case of Example 9, the acetophenone used is prepared according to the method described in Heterocycles 2002, 57, 123-128.

α,2,2′,4,4′,6,6′-heptamethoxychalcone is prepared like in Example 1. The acetophenone used (2,4,6-ω-tetramethoxyacetophenone) is prepared according to the method described in J. Chem. Soc., Perkin Trans. 1. 1991, 3005-3008.

For the whole of these compounds, it was demonstrated by nuclear magnetic resonance that the α,β double bond is trans.

A. Cycle Test

THE CYCLE TEST CORRESPONDS TO THE ANALYSIS OF THE BLOCKING IN THE G2/M PHASE OF A LINE OF HUMAN LEUKEMIC CELLS AS EVALUATED BY FLOW CYTOMETRY.

The k562 cells are incubated for 24 hours in the absence or in the presence of compounds according to the invention and/or reference compounds such as vincristine. In a first phase, the compounds are used at a concentration of 10 μM, whereby dose-response type studies may be carried out secondarily. After 24 hours the cells are exposed to propidium iodide, a DNA intercalating agent, and analyzed in flow cytometry. The results are expressed as a percentage of the cells blocked in the G2/M phase of the cell cycle.

The results shown in TABLE 2 hereafter are obtained with compounds of formula (I) of the invention and are shown as a comparison with the inhibition % of a compound described in the prior art U.S. Pat. No. 6,462,075 (called compound A):

TABLE 2

| | | G2/M inhibition |
|---|---|---|
| Compounds | Formula | in % |
| Described in U.S. Pat. No. 6,462,075 called Compound A | 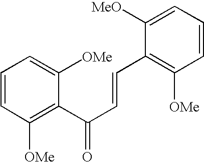 | 78% |
| Example 1 | 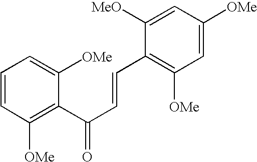 | 84% |
| Example 2 | 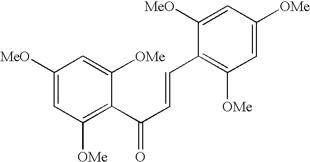 | 86% |
| Example 3 | 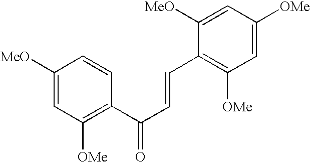 | 86% |

MTT Tests

The MMT test is a metabolic test used for evaluating the inhibition of proliferation of tumoral lines by a compound according to the invention. Several representative tumoral lines of different tumoral types such as breast cancer, colon cancer, otorhinolaryngological tumors, lung cancer, are incubated in a 96-well plate at variable concentrations of a compound according to the invention, for 72 hours and MTT is then added for a period of 1 hour. The MTT is a substrate of mitochondrial enzymes which is degraded in insoluble formazan blue. The crystals are then re-solubilized in isopropanol-HCl 0.1N and the plates are analyzed on an ELISA plate reader. Relative survival is calculated relatively to cells which have not been exposed to the compound according to the invention. The IC₅₀ or inhibiting concentration 50 is the concentration value of the investigated compound according to the invention which induces a 50% reduction of proliferation relatively to the control.

Comparisons of the compounds of Examples 1 and 2 with the compound A were therefore carried out by conducting dose-response studies in flow cytometry and cytotoxicity studies (“MTT tests”). The latter test reflects the capacity of the compounds of destroying tumoral cells. The results shown in FIG. 1 and TABLE 3 show that the compounds of Examples 1 and 2 have better activity than the compound A, both in the flow cytometry test and in the cytotoxicity test:

in FIG. 1 we observe significant G2/M cycle blocking from the concentration of the order of one micromolar for the compounds of Examples 1 and 2 whereas the compound A has no activity at this same concentration;

the cytotoxicity inhibiting concentrations IC₅₀ are about 10 times smaller for the compounds of Examples 1 and 2 than for the compound A in several representative tumoral lines of different types of cancers as shown by the results of in vitro cytotoxicity tests (MTT) as shown in TABLE 3.

TABLE 3

| | | | NIH | | HNO | HCT |
|---|---|---|---|---|---|---|
| IC₅₀ (μM) | MCF7 | N2A | 3T3 | SW48 | 150 | 116 |
| Compound A | 75 | 55 | 60 | 10 | 62 | 9 |
| Example 1 | 60 | 2.2 | 30 | 0.25 | 1.3 | 0.45 |
| Example 2 | 52 | 4 | 30 | 0.8 | 10 | 1 |

| IC₅₀ (μM) | Messa | CEM | K562 | RL | L1210 |
|---|---|---|---|---|---|
| Compound A | 10 | 6 | 80 | 40 | 34 |
| Example 1 | 1 | 0.65 | 50 | 0.8 | 7 |
| Example 2 | 2.2 | 1.9 | 50 | 0.9 | 8.5 |

The study of the structure-activity relationship with the whole of claimed compounds indicates that the nature of the substituents of the phenyl groups and their position are decisive for antimitotic activity.

On the ring A of the chalcone, a methoxy group seems to be the most advantageous among all the investigated substituents and the 2, 4 and 6 positions are the most important for the substitution. The size of the substituent and therefore steric hindrance around this region of the molecule also has an influence, so that methoxy groups are preferred.

More hydrophobic groups such as methoxy also seem more advantageous. Finally, it should be emphasized that trimethoxylation on the three positions (2, 4 and 6) is the most advantageous as compared with dimethoxylation or monomethoxylation on the same positions. The role played by the oxygen atom of the methoxy group is essential because substitution of a methoxy with a methyl causes a decrease of the antimitotic effect. It should be emphasized that maintaining trimethoxylation in 2, 4 and 6 and an electronic perturbation by introducing two fluorine atoms in 3 and 5 also result in a substantial lowering of the activity.

On ring B, the superior role of the methoxy group is also noted there. A methoxylation preferably on positions 2, 4 and 6 induces a good antimitotic effect. The size of the substituent and therefore the steric hindrance around this region of the molecule also have an influence so that methoxy groups are preferred.

More hydrophobic groups such as methoxy also seem more advantageous.

Maintaining two methoxy groups in 2′ and 6′ and the presence of a NH₂ (amino) group in 4′ only induces a slight loss of activity. The presence of NH₂ should allow rapid access to other derivatives of chalcones by simple alkylation or acylation of the nitrogen.

In conclusion, the presence of di- or tri-methoxylation on the 2,4,6 carbon atoms of ring A, and on the 2′,4′,6′ carbon atoms of ring B is particularly advantageous for antimitotic activity of the chalcones described in this invention.

1. Novel derivatives of chalcones of formula (I): 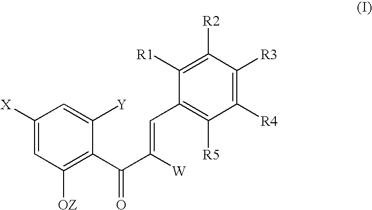
X represents a hydrogen atom or an —OH, —O-methyl, —O-ethyl, —O-propyl, —O-isopropyl, —O-benzyl, —NH₂, —NHCORₐ group
with Rₐ which is a linear or branched alkyl group with 1 to 5 carbon atoms, an aryl group which may be mono- or poly-substituted with a substituent selected from halogen atoms, OH, OMe and —NR_{b}R_{c} groups with R_{b} and R_{c} representing independently of each other a linear or branched alkyl chain with 1 to 6 carbon atoms,
Y represents a hydrogen atom or an —OH, —O-methyl, —O-ethyl, —O-propyl, —O-isopropyl, —O-benzyl group,
it being understood that at least one of the X and Y groups is different from hydrogen,
Z represents a hydrogen atom or a methyl, ethyl, propyl, isopropyl, benzyl group,
W represents a hydrogen atom, or an —H, —O-methyl, —O-ethyl, or —O-benzyl group,
R₁, R2, R3, R3, R4 and R5 represent independently of each other a hydrogen or halogen atom, or an —OH, methyl, —O-methyl, ethyl, —O-ethyl, propyl, —O-propyl, benzyl, —O-benzyl, —NH₂, —NHCH₃, —N(CH₃)₂ group, it being understood that at least two substituents R1, R2, R3, R4 and R5 are different from H, and that when X or Y represents a hydrogen atom, at least three substituents R1, R2, R3, R4 and R5 are different from H,
as well as their pharmaceutically acceptable hydrates, solvates or salts, except for 2′-hydroxy-4′,6′,2,4,5-pentamethoxychalcone, and 2′,4′,6′,2,4,5,6-pentamethoxy-chalcone.

2. The novel chalcone derivatives according to claim 1 characterized in that at least three of the substituants R1, R2, R3, R4 and R5 are different from hydrogen.

3. The novel chalcone derivatives according to claim 1, characterized in that X represents a hydrogen atom or an —OMe, —OEt, —NH₂, group, the OMe group being preferred, it being understood that, when X represents a hydrogen atom, Y represents an —OH, —O-methyl, —O-ethyl, —O-propyl, —O-isopropyl, —O-benzyl group.

4. The novel chalcone derivatives according to claim 1, characterized in that Y represents a hydrogen group or an —OMe, —OEt group, the OMe group being preferred, it being understood that, when Y represents a hydrogen atom, X represents an —OH, —O-methyl, —O-ethyl, —O-propyl, —O-isopropyl, —O-benzyl group.

5. The novel chalcone derivatives according to claim 1, characterized in that Z represents a hydrogen atom or a methyl or ethyl group.

6. The novel chalcone derivatives according to claim 1, characterized in that W represents an —O-methyl group or preferably a hydrogen atom.

7. The novel chalcone derivatives according to claim 1, characterized in that R1, R2, R3, R4 and R5 represent independently of each other, a hydrogen or fluorine atom, a methyl, —O-methyl, —O-ethyl group, it being understood that at least two substituants R1, R2, R3, R4 and R5 are different from H, and that when X or Y represents a hydrogen atom, at least three substituants R1, R2, R3, R4 and R5 are different from H.

8. The novel chalcone derivatives according to claim 1, characterized in that they fit formula (Ia): 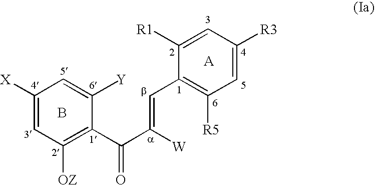
wherein
X represents a hydrogen atom or an —OH, —O-methyl, —O-ethyl, —O-propyl, —O-isopropyl, —O-benzyl, —NH_{2,} —NHCORₐ group
with R₁ which is a linear or branched alkyl group with 1 to 5 carbon atoms, an aryl group which may be mono- or poly-substituted with a substituent selected from halogen atoms, OH, OMe and —NR_{b}R_{c} groups with R_{b} and R_{c} representing independently of each other a linear or branched alkyl chain with 1 to 6 carbon atoms,
Y represents a hydrogen atom or an —OH, —O-methyl, —O-ethyl, —O-propyl, O-isopropyl, —O-benzyl group,
it being understood that at least one of the X and Y groups is different from hydrogen, and
R1, R3, and R5 represent independently of each other a hydrogen or halogen atom, or an —OH, methyl, —O-methyl, ethyl, —O-ethyl, propyl, —O-propyl, benzyl, —O-benzyl, —NH₂, —NHCH₃, —NCH₃₂ group, it being understood that at least two substituents R1, R2, R3, R4 and R5 are different from H, and that when X or Y represents a hydrogen atom, at least three substituents R1, R2, R3, R4 and R5 are different from H,
as well as their pharmaceutically acceptable hydrates, solvates or salts, except for 2′-hydroxy4′,6′,2,4,5-pentamethoxychalcone, and 2′,4′,6′,2,4,5,6-pentamethoxy-chalcone.

9. The novel chalcone derivatives according to claim 1 selected from:
2,2′,4,6,6′-pentamethoxychalcone: 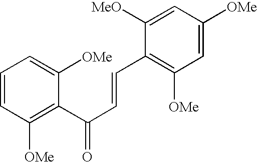
2,2′,4,4′,6,6′-hexamethoxychalcone: 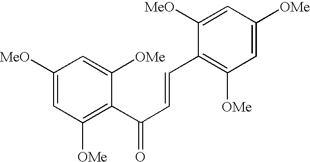
2,2′,4,4′,6-pentamethoxychalcone: 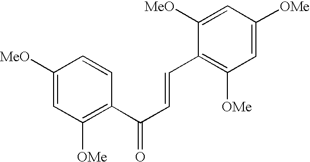
2′-hydroxy-2,4,4′,5-tetramethoxychalcone: 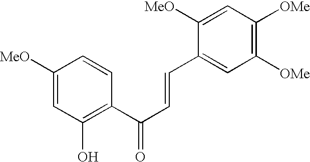
2′-hydroxy-2,4,4′,6-tetramethoxychalcone: 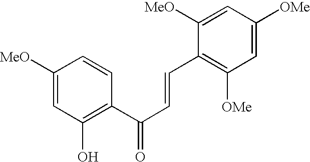
2′-hydroxy-3,4,4′,5-tetramethoxychalcone: 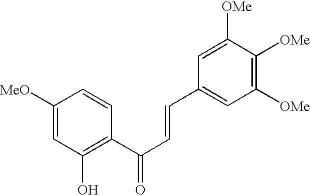
2′-hydroxy-2,3,4,4′-tetramethoxychalcone: 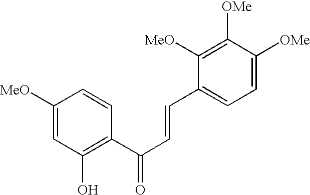
2′,6′-diethoxy-2,4,6-trimethoxychalcone: 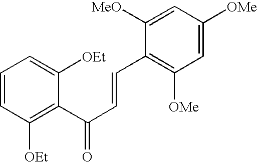
2′,6′-dimethoxy-2,4,6-triethoxychalcone: 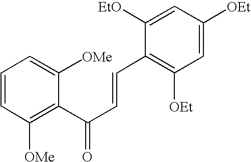
4′-amino-2,2′,4,6,6′-pentamethoxychalcone: 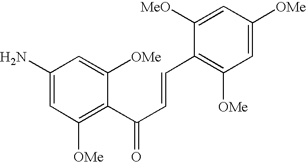
2,2′,4,4′,6,6′-hexaethoxychalcone: 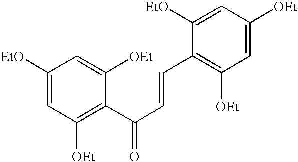
2,2′,3,4,6′-pentamethoxychalcone: 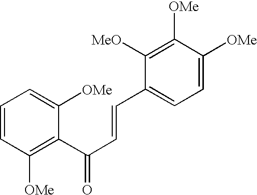
2,2′,4,5,6′-pentamethoxychalcone: 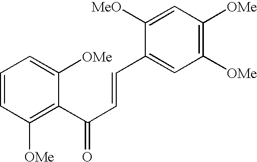
2′,3,4,5,6′-pentamethoxychalcone: 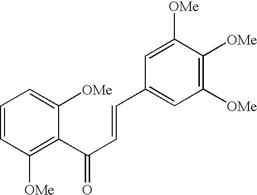
3,5-difluoro-2,2′,4,6,6′-pentamethoxychalcone: 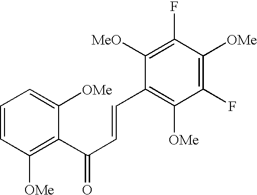
2′,6′-dimethoxy-2,4,6-trimethylchalcone: 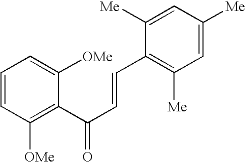
2,2′,4,4′,6′-pentamethoxychalcone: 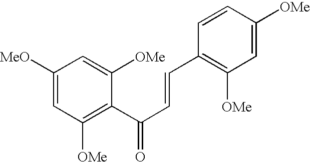
2,2′,4′,6,6′-pentamethoxychalcone: 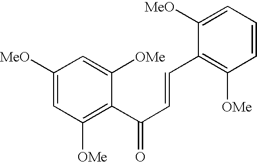
α,2,2′,4,4′,6,6′-heptamethoxychalcone:

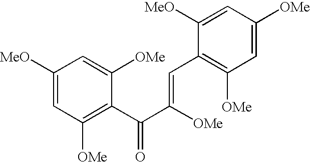

10. The compounds according to claim 1 as a drug.

11. A pharmaceutical composition containing one of the compounds according to claim 1, in association with at least one pharmaceutically acceptable excipient.

12. The use of one of the compounds according to claim 1 for making a drug intended for treating or preventing cancers, including malignant hemopathies and solid tumors, including tumors of glandular, mesenchymatous, genital, cutaneous and neurological origin.

13. The use of one of the compounds according to claim 1 for making a drug intended for treating or preventing benign tumoral lesions.

14. The use of one the compounds according to claim 1 for making a drug intended for treating or preventing immunity disorders characterized by excessive activity of the immune system.

15. The use of one of the compounds according to claim 1 for making a drug intended for treating or preventing pathologies characterized by excessive angiogenic activity.
